(19) United States
(12) Patent Application Publication
(10) Pub. No.: US 2010/0234948 A1
(43) Pub. Date: Sep. 16, 2010

(54) METHODS FOR IMPROVING THE BIOACTIVITY CHARACTERISTICS OF A SURFACE AND OBJECTS WITH SURFACES IMPROVED THEREBY

(75) Inventors: Joseph Khoury, Dedham, MA (US); Laurence B. Tarrant, Cambridge, MA (US); Sean R. Kirkpatrick, Littleton, MA (US); Richard C. Svrluga, Newton, MA (US)

Correspondence Address: BURNS & LEVINSON, LLP, 125 SUMMER STREET, BOSTON, MA, 02110, US

(73) Assignee: EXOGENESIS CORPORATION, Billerica, MA (US)

(21) Appl. No.: 12/722,473

(22) Filed: Mar. 11, 2010

Related U.S. Application Data
(60) Provisional application No. 61/168,971, filed on Apr. 14, 2009.
(60) Provisional application No. 61/218,170, filed on Jun. 18, 2009.
(60) Provisional application No. 61/238,462, filed on Aug. 31, 2009.
(60) Provisional application No. 61/159,113, filed on Mar. 11, 2009.

(51) Int. Cl. A61F 2/02; C12N 11/00; C12N 11/14; C12N 11/08; H01J 37/317

(52) U.S. Cl. 623/11.11; 435/174; 435/176; 435/180; 250/492.3

(57) ABSTRACT

The invention provides for a method of improving bioactivity of a surface of an implantable object. The invention also provides for a method of improving bioactivity of a surface of biological laboratory ware. The invention further provide a method of attaching cells to an object. The invention even further provides for a method of preparing an object for medical implantation. The invention also provides for an article with attached cells, and for an article for medical implantation.

### CROSS REFERENCE TO RELATED APPLICATIONS

This application claims priority of U.S. provisional application Ser. No. 61/168,971 entitled “Methods for Improving the Bioactivity Characteristics of a Surface and Objects with Surfaces Improved Thereby”, filed Apr. 14, 2009, and U.S. provisional application Ser. No. 61/218,170 entitled “Methods for Improving the Bioactivity Characteristics of a Surface and Objects with Surfaces Improved Thereby”, filed Jun. 18, 2009, and U.S. provisional application Ser. No. 61/238,462 entitled “Methods for Improving the Bioactivity Characteristics of a Surface and Objects with Surfaces Improved Thereby”, filed Aug. 31, 2009, and U.S. provisional application Ser. No. 61/159,113 entitled “Methods for Modifying the Wettability and other Biocompatability Characteristics of a Surface of a Biological Material by the Application of Gas Cluster Ion Beam Technology and Biological Materials Made Thereby”, filed Mar. 11, 2009, all of which applications are being incorporated herein by reference.

### FIELD OF THE INVENTION

This invention relates generally to methods for improving the bioactivity characteristics of a surface of an object and to production of objects having at least a portion of a surface with improved bioactivity. More specifically, it relates to methods for improving a surface by increasing its bioactivity through the use of gas-cluster ion-beam technology.

### BACKGROUND OF THE INVENTION

It is often desirable that an object will have a surface that has an increased ability to attract and host the growth, attachment and proliferation of living biological cells. This is often the case for certain biological laboratory wares, including for example, tissue culture dishes, flasks and roller flasks, wells and chamber slides, plates, Petri dishes, etc. It is also often the case for medical objects intended for implant and also for environmental testing devices used to test airborne or waterborne contaminants.

As used herein, the term “bioactivity,” used in relation to a surface or an object or portion of an object, is intended to mean suitability of the surface or object or object portion for attracting living cells thereto, or for improving cell and/or tissue activity thereon, or for attaching living cells thereto, or for promoting growth of living cells thereon, or for promoting proliferation of living cells thereon. As used herein the term “titania” is intended to include oxides of titanium in all forms including ceramic forms, and the titanium metal itself (or an alloy thereof) together with a surface coating of native oxide or other oxide comprising the element titanium (including without limitation TiO₂, and or TiO₂ with imperfect stoichiometry). Implantable medical devices are often fabricated from titanium metal (or alloy) that typically has a titania surface (which may be either a native oxide, or a purposely oxidized surface, or otherwise).

Biological laboratory wares may be employed in cell culture, tissue culture, explant culture, and tissue engineering applications (for examples) and is commonly formed from generally inert and/or biocompatible materials like glass, quartz, plastics and polymers, and certain metals and ceramics. It is often desirable to be able to modify at least a portion of the surface of such biological laboratory wares to enhance their bioactivity.

Medical objects intended for implant into the body or bodily tissues of a mammal (including human), as for example medical prostheses or surgical implants or grafts, may be fabricated from a variety of materials including, but not limited to, various metals, metal alloys, plastic or polymer or co-polymer materials (including woven, knitted, and non-woven polymeric/co-polymeric fabrics), solid resin materials, glass and glassy materials, biological materials such as bone and collagen, silk and other natural fibers, and other materials (including without limitation, poly[glutamic acid], poly[lactic-co-glycolic acid], and poly[L-lactide]) that may be suitable for the application and that are appropriately biocompatible. As examples, certain stainless steel alloys, titanium and titanium alloys (including possible native oxide coatings), cobalt-chrome alloys, cobalt-chrome-molybdenum alloy, tantalum, tantalum alloys, zirconium, zirconium alloys (including possible native oxide coatings), polyethylene and other inert plastics, and various ceramics including titania, alumina, and zirconia ceramics are employed. Polymeric/co-polymeric fabrics may for example be formed from polyesters (including polyethylene terephthalate(PETE)), polytetrafluoroethylene (PTFE), aramid, polyamide or other suitable fibers. Medical objects intended for implant include for example, without limitation, vascular stents, vascular and other grafts, dental implants, artificial and natural joint prostheses, coronary pacemakers, implantable lenses, etc. and components thereof. Often such a device may have a native surface state with cellular adhesion and cellular proliferation properties that are less than ideal for the intended purpose. In such cases it is often desirable to be able to modify at least a portion of the surface of the object to enhance cellular attachment thereto in order to make it more suitable for the implant application.

Environmental testing devices often include materials such as metals, plastics and polymers, glasses and quartz, etc.

Gas-cluster ion-beam (GCIB) irradiation has been used for nano-scale modification of surfaces. In the co-pending, commonly held U.S. patent application Ser. No. 12/210,018, “Method and System for Modifying the Wettability Characteristics of a Surface of a Medical Device by the Application of Gas Cluster Ion Beam Technology and Medical Devices Made Thereby,” GCIB irradiation has been shown to modify the hydrophilic properties of non-biological material surfaces. It is generally known that cells, including but not limited to, anchorage-dependent cells such as fibroblasts and osteoblasts prefer hydrophilic surfaces to attach, grow, or differentiate well and they also prefer charged surfaces at physiological pH. Many methods have been employed to increase hydrophilicity or alter charge on non-biological surfaces, such as sandblasting, acid etching, sandblasting plus acid etching (SLA), plasma spraying of coatings, CO₂ laser smoothing and various forms of cleaning, including mechanical, ultrasonic, plasma, and chemical cleaning techniques. Other approaches have included the addition of surfactants or the application of films or coatings having different wettability characteristics. Various methods have also been employed to increase cell adherence properties of surfaces such as UV treatment, UV and ozone treatment, covalently attaching poly(ethylene glycol) (PEG), and the application of protein products such as the antibody anti-CD34 and arginine-glycine-aspartate peptides (RGD peptides).

It is therefore an object of this invention to provide a surface and an object having at least a portion of its surface modified by GCIB processing to have improved bioactivity.

It is further an objective of this invention to provide methods of forming a surface or an object having at least a portion of its surface modified to have improved bioactivity by employing GCIB technology.

Yet another objective of this invention is to provide an object for medical implantation having at least a portion of its surface modified by GCIB processing and having cells attached in vitro prior to medical implantation.

A still further objective of this invention is to provide methods of forming an object for medical implantation having at least a portion of its surface modified by GCIB technology and by in vitro attachment of cells prior to medical implantation.

### SUMMARY OF THE INVENTION

The objects set forth above as well as further and other objects and advantages of the present invention are achieved by the invention described herein below.

One of the fundamental challenges in tissue engineering has been the ability to allow cells from different lineages to grow and interact in a manner seen in the human body. GCIB irradiation of surfaces greatly improves cell adherence and proliferation while maintaining cellular differentiation. Wound repair in tissues and organs derived from epithelial, endothelial, mesenchymal, or neuronal cells can benefit when they are grown on inert or bio-active material that has been surface modified by GCIB irradiation. Whether the goal is to achieve integration between underlying bone and a dental implant; cellular infiltration and integration between a ligament and the attaching bone; enhancing skin or hair graft integration; or nerve regeneration to re-initiate synapses, the use of GCIB irradiation is a useful process in the progression of tissue engineering and wound repair.

The present invention is directed to the use of GCIB processing to form surface regions on objects intended for cellular attachment, the surface regions having improved bioactivity properties to facilitate growth, attachment and/or proliferation of cells. It is also directed to the in vitro attachment of cells to the GCIB processed surface regions of medical objects prior to medical/surgical implantation. The attached cells may be derived from the body of the individual for whom the medical/surgical implant is intended or may be derived from other compatible sources.

When it is intended that certain selected portions of the surface of the object intended for cell attachment should have improved bioactivity properties, and when it is intended that other portions of the surface of the object not be involved in cell attachment processes, GCIB processing may be limited to the selected portions by limiting the GCIB processing to only the selected portions of the surface of the object to increase the bioactivity properties for only the selected portions. Controlling the GCIB cross-sectional area and/or controlling the scanning and/or deflecting of the GCIB to limit the extent of its irradiation to only the selected surface portions may accomplish the limitation of GCIB processing to selected regions. Alternatively, conventional masking technology may be used to mask the surface portions for which GCIB processing is not desired, and to expose the selected surface portions for which GCIB processing is required. Subsequently the mask and the surface portions exposed through the mask may be irradiated with a diffuse or scanned GCIB. Various other methods of limiting the GCIB irradiation to selected regions of a surface or of the surface of an object will be known to those skilled in the art and are intended to be encompassed in the invention.

Beams of energetic conventional ions, accelerated electrically charged atoms or molecules, are widely utilized to form semiconductor device junctions, to modify surfaces by sputtering, and to modify the properties of thin films. Unlike conventional ions, gas-cluster ions are formed from clusters of large numbers (having a typical distribution of several hundreds to several thousands with a mean value of a few thousand) of weakly bound atoms or molecules of materials that are gaseous under conditions of standard temperature and pressure (commonly oxygen, nitrogen, or an inert gas such as argon, for example, but any condensable gas can be used to generate gas-cluster ions) with each cluster sharing one or more electrical charges, and which are accelerated together through high voltages (on the order of from about 3 kV to about 70 kV or more) to have high total energies. After gas-cluster ions have been formed and accelerated, their charge states may be altered or become altered (even neutralized), and they may fragment into smaller cluster ions and/or neutralized smaller clusters, but they tend to retain the relatively high total energies that result from having been accelerated through high voltages. Being loosely bound, gas-cluster ions disintegrate upon impact with a surface and the total energy of the accelerated gas-cluster ion is shared among the constituent atoms. Because of this energy sharing, the atoms in the clusters are individually much less energetic (after disintegration) than as is the case for conventional ions and, as a result, the atoms penetrate to much shallower depths, despite the high energy of the accelerated gas-cluster ion. As used herein, the terms “GCIB”, “gas-cluster ion-beam” and “gas-cluster ion” are intended to encompass accelerated beams and ions that have had all or a portion of their charge states modified (including neutralized) following their acceleration. The terms “GCIB” and “gas-cluster ion-beam” are intended to encompass all beams that comprise accelerated gas clusters even though they may also comprise non-clustered particles.

Because the energies of individual atoms within a gas-cluster ion are very small, typically a few eV to some tens of eV, the atoms penetrate through, at most, only a few atomic layers of a target surface during impact. This shallow penetration (typically a few nanometers to about ten nanometers, depending on the beam acceleration) of the impacting atoms means all of the energy carried by the entire cluster ion is consequently dissipated in an extremely small volume in a very shallow surface layer during a time period of less than a microsecond. This differs from conventional ion beams where the penetration into the material is sometimes several hundred nanometers, producing changes and material modification deep below the surface of the material. Because of the high total energy of the gas-cluster ion and extremely small interaction volume, the deposited energy density at the impact site is far greater than in the case of bombardment by conventional ions. Accordingly, GCIB processing of a surface can produce modifications that can enhance properties of the surface to result in improved suitability for subsequent cell growth, attachment and proliferation.

Without wishing to be bound to any particular theory, it is believed that the increased bioactivity observed for surfaces processed by GCIB irradiation according to the methods of the invention may result from a physical transformation of the structure of the GCIB irradiated surfaces.

Gas-cluster ion beams are generated and transported for purposes of irradiating a workpiece according to known techniques as taught for example in the published U.S. Patent Application 2009/0074834A1 by Kirkpatrick et al., the entire contents of which are incorporated herein by reference. Essential steps include injecting a high pressure gas into a reduced-pressure chamber to form a jet where gas clusters form during expansion of the gas, separating the gas clusters from most of the unclustered gas in the jet, ionizing the gas clusters to form gas-cluster ions, and forming, accelerating, and directing a beam of the gas-cluster ions onto workpieces in the reduced-pressure environment for processing by GCIB irradiation. The workpiece may be introduced into the reduced pressure chamber prior to evacuating the chamber or through atmosphere-vacuum load locks by techniques known to those skilled in the art. Various types of holders are known in the art for holding the object in the path of the GCIB for irradiation and for manipulating the object to permit irradiation of a multiplicity of portions of the object.

The objects having GCIB improved surfaces according to the invention may be employed (for example, not for limitation) in biological laboratory wares intended for cell culture, tissue culture, explant culture, tissue engineering, or other cell attachment or growth applications) or may be medically/surgically implanted into or onto the body or bodily tissues of a mammal or other biological entity, or may be employed for environmental testing applications, etc. Optionally, objects may be additionally processed to effect in vitro attachment of cells onto the GCIB processed surfaces prior to their application, as in for example, medical/surgical implantation.

The invention provides for a method of improving bioactivity of a surface of an implantable object. The method comprises the steps of forming a gas-cluster ion-beam in a reduced-pressure chamber, introducing an object into the reduced-pressure chamber; and irradiating at least a first portion of the surface of said object with a gas-cluster ion-beam. The object on the method is a medical prosthesis, a surgical implant, a surgical graft, a component of a medical prosthesis, a component of a surgical implant, a component of a surgical graft, or another object intended for implantation.

The invention also provides for a method of improving bioactivity of a surface of biological laboratory ware. The method comprises the steps of forming a gas-cluster ion-beam in a reduced-pressure chamber, introducing an object into the reduced-pressure chamber, and irradiating at least a first portion of the surface of the object with a gas-cluster ion-beam. The object of the method is an item of biological laboratory ware.

The invention further provide for a method of attaching cells to an object. The method comprises the steps of selecting at least a portion of a surface of an object, forming a gas-cluster ion-beam in a reduced-pressure chamber, introducing said object into said reduced-pressure chamber, irradiating said at least a portion of said surface with said gas-cluster ion-beam, removing said object from said reduced-pressure chamber, and exposing said at least a portion of said surface to living cells.

The invention even further provide for a method of preparing an object for medical implantation, The method comprises the steps of selecting at least a portion of a surface of an object, forming a gas-cluster ion-beam in a reduced-pressure chamber, introducing the object into the reduced-pressure chamber, and irradiating the selected at least a portion with the gas-cluster ion-beam to increase the bioactivity of the at least a portion. The object of the method is a medical implant.

The invention still further provides for an article with attached cells made by a method comprising the steps of selecting at least a portion of a surface of an object for attaching cells, forming a gas-cluster ion-beam in a reduced-pressure chamber, introducing said article into said reduced-pressure chamber, irradiating said at least a portion of said surface with the gas-cluster ion-beam, removing said object from said reduced-pressure chamber, and exposing said at least a portion of said surface to living cells.

The invention yet further provide for nn article for medical implantation made by a method comprising the steps of selecting at least a portion of a surface of a medical implant, forming a gas-cluster ion-beam in a reduced-pressure chamber, introducing said implant into said reduced-pressure chamber; and irradiating said at least a portion of said surface with the gas-cluster ion-beam to increase the bioactivity of said at least a portion of said surface.

### BRIEF DESCRIPTION OF THE DRAWINGS

For a better understanding of the present invention, together with other and further objects thereof, reference is made to the accompanying drawings, wherein:

FIG. 1 is a chart 100 comparing rates of cellular attachment and proliferation

FIG. 2 is a scanning electron micrograph 200 of a portion of a surface of an untreated titanium foil showing attachment of cells to the surface;

FIG. 3 is a scanning electron micrograph 300 of a portion of a surface of a titanium foil processed by GCIB irradiation according to an embodiment of the invention showing improved attachment/proliferation of cells to the surface;

FIGS. 4a through 4f are optical micrographs of portions of surfaces of glass substrates, both controls and GCIB irradiated, according to an embodiment of the invention and showing improved attachment/proliferation of cells on the surface following GCIB irradiation;

FIGS. 5a through 5i are optical micrographs of portions of surfaces of polystyrene substrates, including controls, GCIB irradiated, and commercial cell culture processed, according to an embodiment of the invention and showing improved attachment/proliferation of cells on the surface having received GCIB irradiation;

FIGS. 6a and 6b are optical micrographs of portions of a surface of a polystyrene substrate, wherein a portion of the surface was masked during GCIB irradiation, so as to show side-by-side comparison of the un-irradiated masked portion with the GCIB irradiated portion and showing improved attachment/proliferation of cells on the GCIB irradiated portion;

FIGS. 7a and 7b are electron micrographs of portions of surfaces of PTFE substrates, wherein FIG. 7a shows a non-ion-beam-irradiated control portion and FIG. 7b shows an GCIB irradiated portion and wherein the GCIB irradiated portion shows significantly improved cellular attachment and/or proliferation in comparison to the control portion;

FIG. 8 is an optical micrograph of portions of a surface of an amorphous quartz substrate, wherein a portion of the surface was masked during GCIB irradiation, so as to show side-by-side comparison of the un-irradiated masked portion with the GCIB irradiated portion and showing a high degree of attachment/proliferation of cells on both the GCIB irradiated portion and the un-irradiated portions;

FIG. 9 is an optical micrograph of portions of a surface of a crystalline sapphire substrate, wherein a portion of the surface was masked during GCIB irradiation, so as to show side-by-side comparison of the un-irradiated masked portion with the GCIB irradiated portion and showing a high degree of attachment/proliferation of cells on the GCIB irradiated portion; and

FIG. 10 is a scanning electron micrograph of portions of a surface of a PETE fabric surface, wherein a portion of the fabric surface was masked during GCIB irradiation so as to show side-by side comparison of the un-irradiated masked portion with the GCIB irradiated portion and showing preferential attachment of cells to the GCIB irradiated portion.

### DETAILED DESCRIPTION OF THE PREFERRED METHODS AND EXEMPLARY EMBODIMENTS

Several exemplary embodiments are disclosed to show the wide scope and variety of material surfaces that can enjoy benefit of the GCIB processing method of the invention to enhance their bioactivity. These examples are chosen to illustrate that the application of the invention is broad and not limited to one or a few materials, but can be broadly exploited for a wide range of material surfaces.

### Titanium Exemplary Embodiment

A titanium surface improvement is disclosed in a first exemplary embodiment. Titanium is a material often employed in medical objects intended for implantation into a mammal. Titanium foil samples of 0.01 mm thickness were first cleaned in 70% isopropanol for 2 hours and then air dried in a bio-safety cabinet overnight. It is understood that the cleaned titanium foil samples, as with any titanium that has been exposed to normal atmospheric conditions, likely has a very thin native titania surface coating, which may be incomplete and may be imperfect. The foil samples were then either GCIB irradiated to a dose of 5×10¹⁴ ions/cm² using an argon GCIB accelerated using 30 kV acceleration voltage or were left un-irradiated, as controls. The titanium foils (both the irradiated sample and control sample) were then cut into 0.9 cm×0.9 cm squares and placed at the bottom of individual wells (8 control squares and 8 GCIB irradiated squares) of a 24-well Multiwell™ polystyrene plate (BD Falcon 351147). Human fetal osteoblastic cells derived from bone (hFOB 1.19, ATCC CRL-11372) were sub-cultured and approximately 3500 cells were placed on top of each titanium foil square in 1 ml of (Invitrogen Corp.) Dulbecco's Modified Eagle Medium nutrient mixture F-12 (DMEMIF12) supplemented with 10% fetal bovine serum (FBS) and 0.3 mg/ml G418 antibiotic (also known as Geneticin) and incubated in a humidified incubator at 37° C. and 5% CO² in air. Following one day and five days of incubation, media samples were removed and cells were assayed using CellTiter 96® AQueous Cell Proliferation Assay from Promega used according to the manufacturer's instructions, with the measurement made using a Dynex OpsysMR plate reader at 490 nm wavelength. Assay solution was then removed from the wells and the titanium foils and the cells Were then fixed by placing −20° C. chilled methanol on the titanium foil squares in the wells for at least 30 minutes. Following fixation, the titanium foil squares were then air-dried and osteoblast cells adhering to the titanium foil squares were imaged using a Hitachi TM1000 scanning electron microscope. Results showed that osteoblast cells adhered to the foils following one day of incubation were 694.5 cells +/−164.8 cells on the control foils and were enhanced to 2082.3 cells +/−609.2 cells on GCIB irradiated foils (P<0.03). The osteoblast cells proliferated and after five days incubation were 1598.7 cells +/−728.4 cells on controls as compared to 3898.0 cells +/−940.9 cells on GCIB irradiated foils (P<0.003).

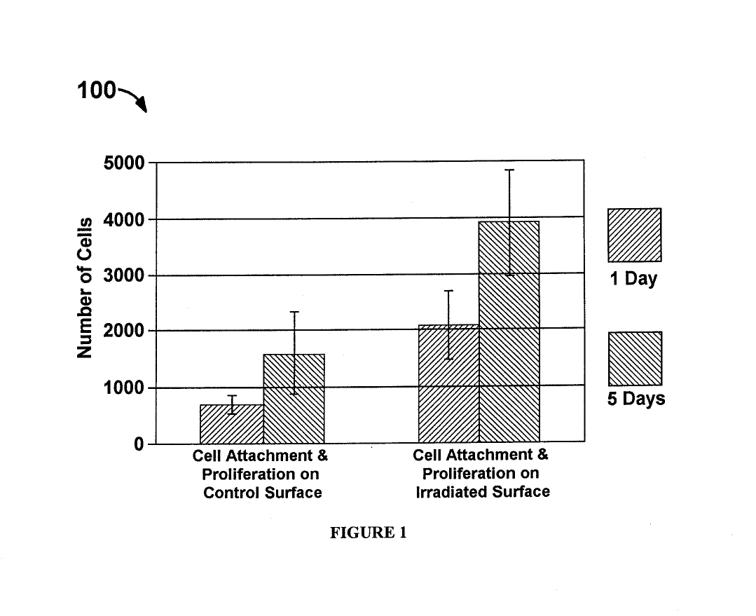

FIG. 1 is a chart 100, which shows that hFOB 1.19 human fetal osteoblastic cells attach to and proliferate at an enhanced rate on GCIB irradiated titanium foils as compared to control titanium foils.

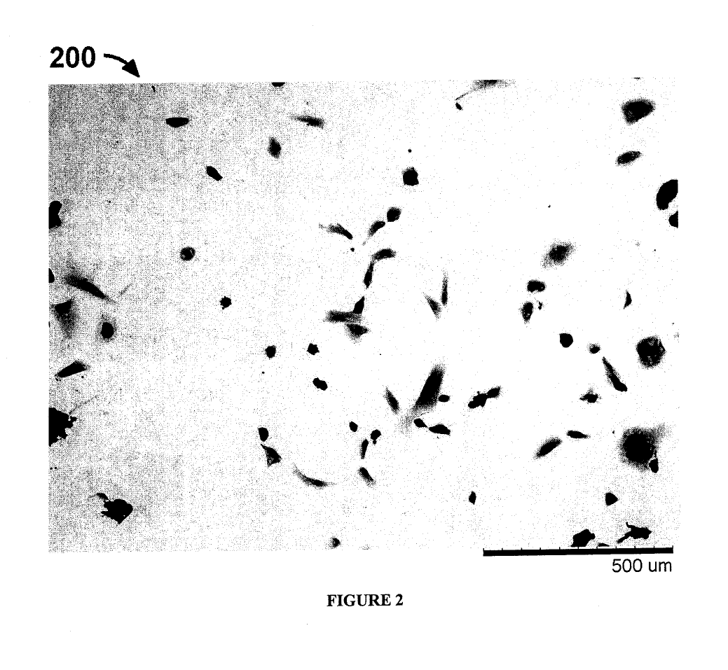

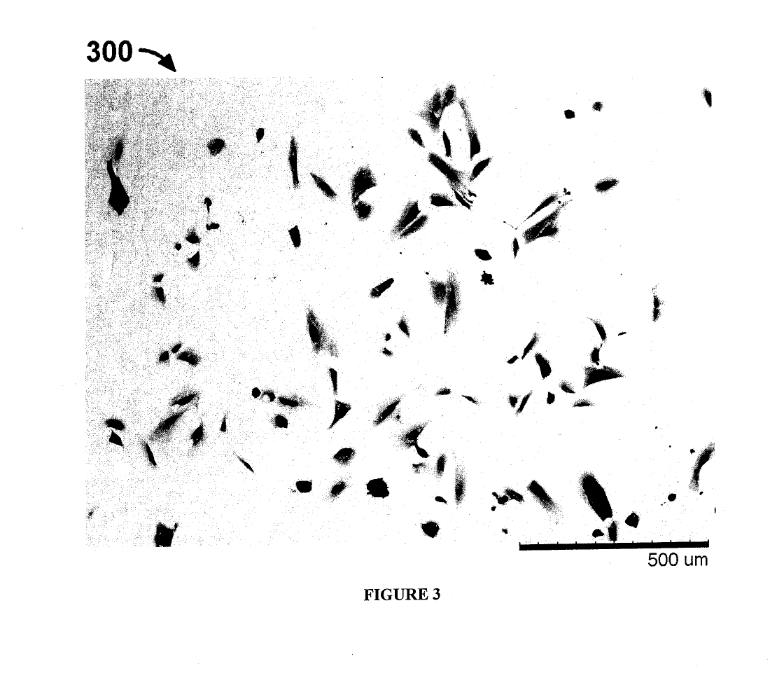

FIG. 2 is a scanning electron micrograph 200 of a control titanium foil following 5 days incubation. FIG. 3 is a scanning electron micrograph 300 of a GCIB irradiated titanium foil following 5 days incubation. Both FIG. 2 and FIG. 3 are shown at the same magnification and image equal surface areas. Comparison of FIG. 2 and FIG. 3 shows that the GCIB irradiated titanium foil (FIG. 3) has an increased degree of osteoblast cell attachment and that more osteoblast cells appear to be spreading and making cell-to-cell contact, which is known to be an important factor in initiating cell proliferation amongst anchorage-dependent cells such as osteoblasts and fibroblasts. GCIB irradiation of materials (such as titanium) employed in forming objects for medical/surgical implantation into a body of a mammal results in modification of the surface to make it more conducive to cell attachment and proliferation.

Employing this effect for improving the integration of a medical object intended for implant into a body or bodily tissue or onto a body of a mammal by making a surface of the object more conducive to cell attachment and proliferation involves the steps of 1) identifying an object for implant wherein it is desired to provide enhanced integration; 2) determining if all surfaces of the object require such enhancement or if it is preferable to limit the enhancement to only a portion of the surfaces of the object (as for example, a hip joint prosthesis wherein the portions that attach to bone benefit from improved attachment while the sliding portion of the ball or acetabular cup do not benefit from increased cellular attachment); and 3) GCIB irradiating only the portions of the surface of the medical object where enhanced integration is desired, and finally medically/surgically implanting the object (modified for enhanced integration) into the body of a mammal. Of course, if it is preferable that all portions of the surface of the medical object benefit from enhanced integration, then all portions of the surface are preferably GCIB irradiated.

Optionally, following the irradiation step and preceding the implanting step, integration may be further enhanced by including a step of growing and attaching (in vitro) cells onto the surface of the medical object. This may include isolating, culturing and in vitro attachment of cells from the particular individual in which the medical object is intended to be implanted, or it may include using cells obtained from another individual, or from stem cells or other pluripotent cells (from either the same or a differing species of mammal).

The irradiating step may optionally include the use of a mask or directed beam or other method for limiting GCIB processing to a selected portion of the object.

In the prior art, micro-roughened titanium surfaces have been shown to be preferential to osteoblast cell attachment. SLA titanium has been a commonly employed material for bone implants. The SLA process both improves the hydrophilicity and micro-roughens the surface. SLA titanium and control (smooth machined) titanium samples were compared, both with and without GCIB irradiation.

Titanium samples (1 cm×1 cm×0.6 mm), with both smooth-machined and SLA surfaces were compared, both with and without argon GCIB irradiation. The smooth-machined and SLA surfaces were characterized for roughness by atomic force microscope measurement techniques. Evaluated over 1-micrometer square scan areas, the average roughness (Ra) values of the two types of surfaces are shown in Table 1.

TABLE 1

| Titanium Sample | Ra (nm) |
|---|---|
| Smooth-Machined | 8.38 |
| SLA surface | 20.08 |

The smooth-machined and SLA surfaces were either irradiated with GCIB at a dose of 5×10¹⁴ argon clusters/cm² at 30 kV acceleration voltage, or left un-irradiated as controls. The titanium pieces (9 samples for each condition, a total of 36 samples) were placed in individual wells in 24 well dishes and approximately 2500 primary human osteoblast cells were placed on each titanium sample in 1 ml of (Invitrogen Corp.) Dulbecco's Modified Eagle Medium nutrient mixture (DMEM) supplemented with 10% fetal bovine serum (FBS) and 1% penicillin/streptomycin and incubated in a humidified incubator at 37° C. and 5% CO₂ in air. Following three days, seven days, and ten days of incubation, three samples for each condition were removed from the media and cells were assayed using CellTiter 96® AQueous Cell Proliferation Assay from Promega used according to the manufacturer's instructions, with the measurement made using a Dynex OpsysMR plate reader at 490 nm wavelength to assess cell attachment to the samples. Results are shown in Table 2.

TABLE 2

| | Cells Attached | | |
|---|---|---|---|
| | (average of three samples) | | |
| Sample Type | 3 Days | 7 Days | 10 Days |
| Smooth-Machined, | 2400 | 7633 | 7567 |
| Un-irradiated | | | |
| Smooth-Machined, | 3767 | 13600 | 17967 |
| GCIB irradiated | | | |
| SLA, Un-irradiated | 3800 | 7333 | 8100 |
| SLA, GCIB irradiated | 2767 | 7467 | 11700 |

The results shown in Table 2 show that little difference existed in cell proliferation between the un-irradiated smooth-machined and un-irradiated SLA titanium surfaces. On the other hand, it is seen that in both cases (smooth-machined and SLA surfaces) the proliferation was substantially enhanced on the GCIB irradiated surfaces. Furthermore, the improvement in proliferation was significantly greater on the smooth-machined (Ra=8.38 nm) surface as compared to the SLA (Ra=20.08 nm) surfaces. It is apparent that though micro-roughness from the SLA process has been considered a preferred surface condition for cell attachment and proliferation in the past, the GCIB irradiation provides superior results even at low roughness values (Ra<10 nm).

### Glass Exemplary Embodiment

A glass surface improvement is disclosed in a second exemplary embodiment. Glass is a material often employed in biological laboratory wares. Glass and glassy or glass-like materials are also employed in fabricating medical objects intended for implantation into a mammal. Thin glass substrates in the form of glass cover slips (Corning Glass 2865-25) were first cleaned in 70% isopropanol for 2 hours and then air-dried. The glass samples were then either GCIB irradiated to a dose of 5×10¹⁴ ions/cm² using an argon GCIB accelerated using 30 kV acceleration voltage or were left un-irradiated, as controls. The glass cover slips (both the irradiated sample and control sample) were then seeded with primary human osteoblast cells at an initial density of 40,000 cells per cm² in Dulbecco's Modified Eagle Medium nutrient mixture (DMEM) supplemented with 10% fetal bovine serum (FBS) and 1% penicillin/streptomycin and incubated in a humidified incubator at 37° C. and 5% CO₂ in air. The glass cover slips were viewed and imaged (optical microscopy) hourly for the first 4 hours to observe cellular attachment. After 4 hours, the nutrient mixture and non-adhering cells were then removed and replaced with fresh, supplemented, nutrient mixture and incubation was continued. Additional microscopic images were taken at 24 hours and 48 hours after seeding.

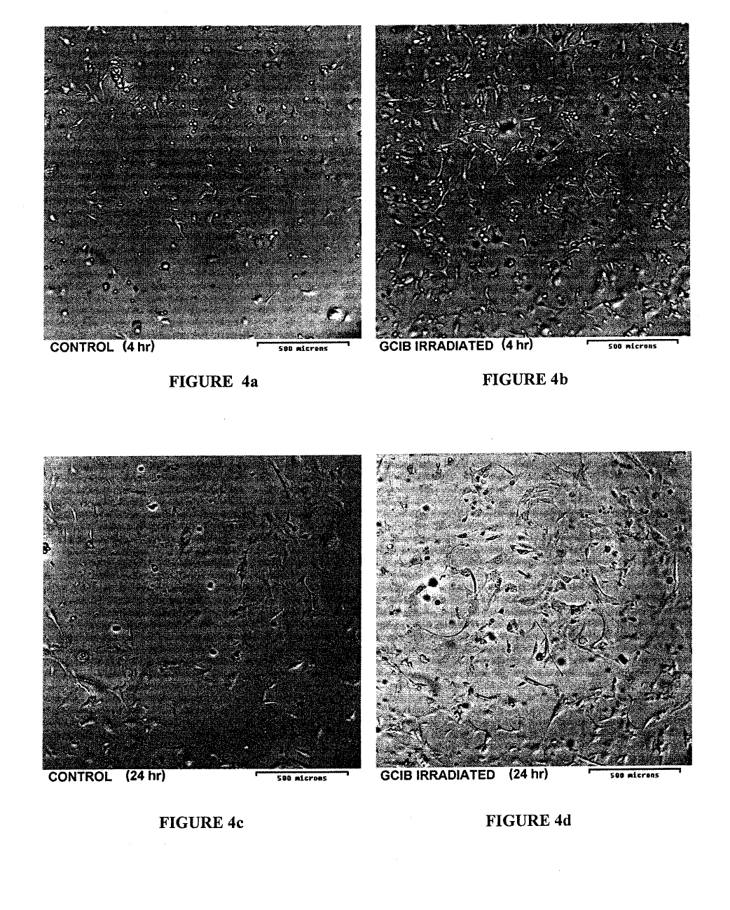

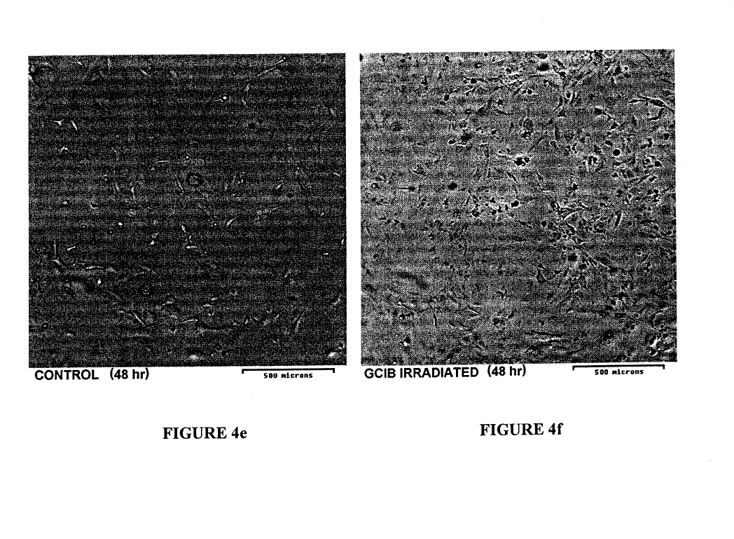

FIGS. 4a, 4c, and 4e are optical micrographs of the control glass cover slip taken at intervals of 4 hours, 24 hours, and 48 hours (respectively) after seeding with cells. FIGS. 4b, 4d, and 4f are optical micrographs of the GCIB irradiated glass cover slip also taken at intervals of 4 hours, 24 hours, and 48 hours (respectively) after seeding with cells. By comparing the controls with the GCIB irradiated surfaces at each time point, it is clear that the human fetal osteoblastic cells attach in greater numbers and proliferate better on the GCIB irradiated glass cover slip surface, compared to the un-irradiated controls.

### Polymer Exemplary Embodiments

A first polymer surface improvement is disclosed in a third exemplary embodiment. Polymer material is a material often employed in biological laboratory wares, for example polystyrene, polypropylene, etc. Polymer materials are also employed in fabricating medical objects intended for implantation into a mammal. Polystyrene substrates in the form of Petri dishes (Fisher Scientific Fisherbrand 08-757-12) were either GCIB irradiated to a dose of 5×10¹⁴ ions/cm² using an argon GCIB accelerated using 30 kV acceleration voltage or were left un-irradiated, as controls. Additionally, a polystyrene substrate in the form of a cell culture dish (BD Biosiences 353003) was employed as an alternative polystyrene surface, for comparison. The cell culture dishes are commercially supplied with a specially treated surface intended to enhance cell growth. The three polystyrene samples (both the irradiated Petri dish sample and control Petri dish sample, as well as the un-irradiated alternative cell culture dish) were then seeded with primary human osteoblast cells at an initial density of 2,500 cells per cm² in Dulbecco's Modified Eagle Medium nutrient mixture (DMEM) supplemented with 10% fetal bovine serum (FBS) and 1% penicillin/streptomycin and incubated in a humidified incubator at 37° C. and 5% CO₂ in air. The three polystyrene samples were viewed and imaged (optical microscopy) hourly for the first 4 hours to observe cellular attachment. After 4 hours, the nutrient mixture and non-adhering cells were then removed and replaced with fresh, supplemented, nutrient mixture and incubation was continued. Additional microscopic images were taken at 24 hours and 48 hours after seeding.

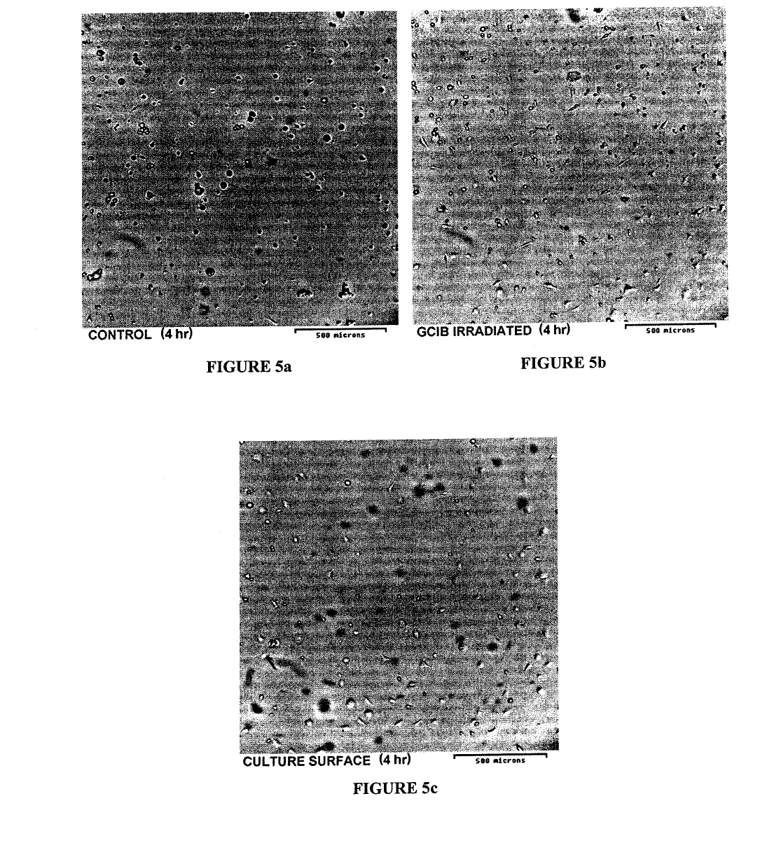

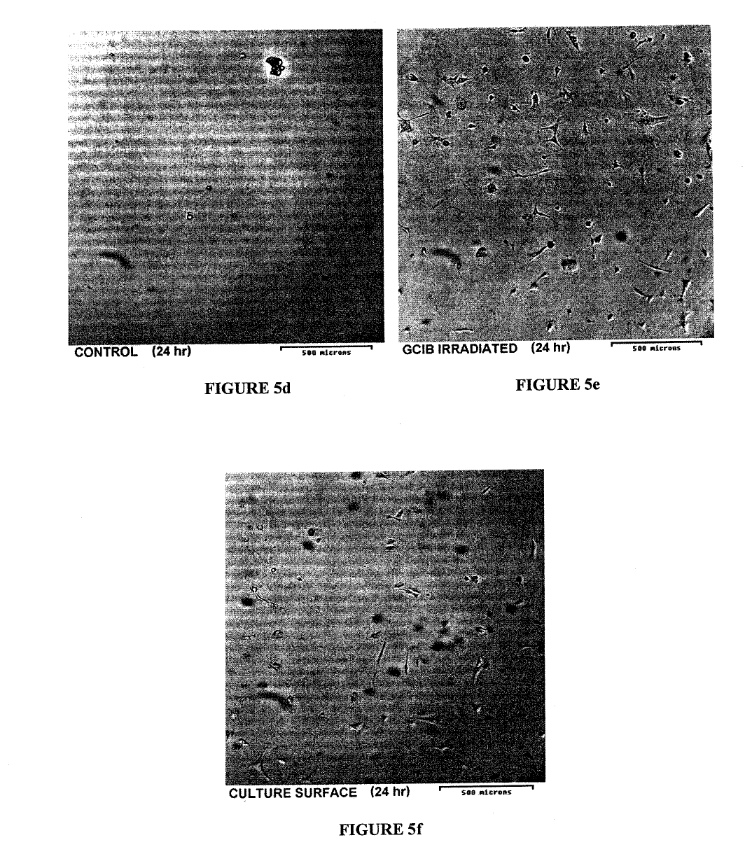

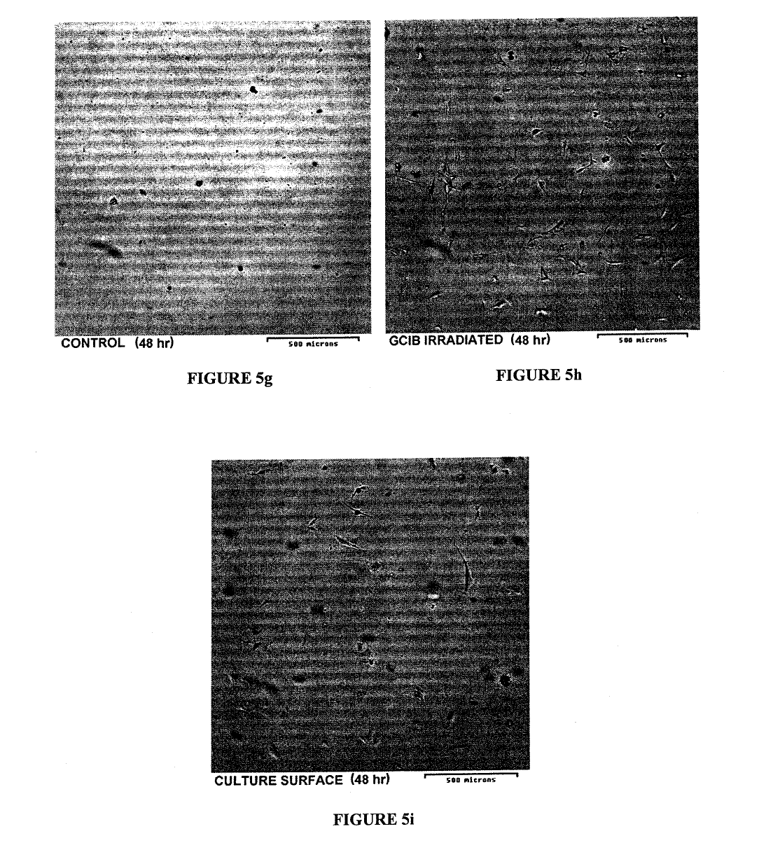

FIGS. 5a, 5d, and 5g are optical micrographs of the surface of the control polystyrene Petri dish taken at intervals of 4 hours, 24 hours, and 48 hours (respectively) after seeding with cells. FIGS. 5b, 5e, and 5h are optical micrographs of the GCIB irradiated polystyrene Petri dish also taken at intervals of 4 hours, 24 hours, and 48 hours (respectively) after seeding with cells. FIGS. 5c, 5f, and 5i are optical micrographs of the GCIB irradiated polystyrene cell culture dish, again taken at intervals of 4 hours, 24 hours, and 48 hours (respectively) after seeding with cells. By comparing the Petri dish control with the GCIB irradiated Petri dish surface and the surface of the un-irradiated cell culture dish at each time point, it is clear that the human fetal osteoblastic cells attach in greater numbers and proliferate better on the GCIB irradiated glass cover slip surface, compared to either the un-irradiated Petri dish control or the un-irradiated cell culture dish surface.

A further polystyrene substrate in the form of a Petri dish (Fisher Scientific Fisherbrand 08-757-12) was partially masked and then GCIB irradiated to a dose of 5×10¹⁴ ions/cm² using an argon GCIB accelerated using 30 kV acceleration voltage. The mask employed was a non-contact shadow mask in proximity to the polystyrene surface. The unmasked portion received the full GCIB dose, while the masked portion received no GCIB irradiation, thus serving as a control surface. The Petri dish was then seeded with primary human osteoblast cells at an initial density of 2,500 cells per cm² in Dulbecco's Modified Eagle Medium nutrient mixture (DMEM) supplemented with 10% fetal bovine serum (FBS) and 1% penicillin/streptomycin and incubated in a humidified incubator at 37° C. and 5% CO₂ in air. The polystyrene Petri dish was viewed (optical microscopy at the interface between the GCIB irradiated and un-irradiated regions) hourly for the first 4 hours to observe cellular attachment. After 4 hours, the nutrient mixture and non-adhering cells were then removed and replaced with fresh, supplemented, nutrient mixture and incubation was continued. Microscopic images were taken at 24 hours and 48 hours after seeding.

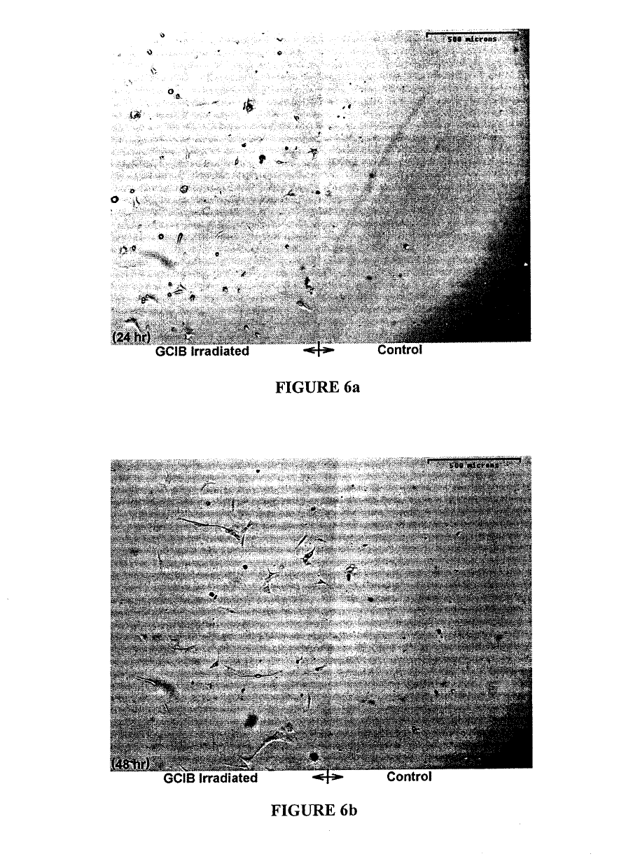

FIGS. 6a, and 6b are optical micrographs of the partially masked polystyrene Petri dish taken at intervals of 24 hours, and 48 hours (respectively) after seeding with cells and viewed at the interface between the masked un-irradiated and the unmasked GCIB irradiated regions. The GCIB irradiated region is on the left side of each of FIGS. 6a and 6b and the un-irradiated control region is on the right side of each of FIGS. 6a and 6b. By comparing the un-irradiated and the GCIB irradiated regions at both time points, it is clear that the human fetal osteoblastic cells attach in greater numbers and proliferate better on the GCIB irradiated portion of the polystyrene surface, compared to the un-irradiated (masked) portion.

A second polymer surface improvement is disclosed in a fourth exemplary embodiment. Polytetrafluoroethylene (PTFE) substrates in the form of strips (30 mm long ×10 mm wide ×1.5 mm thick) were masked on one half and GCIB irradiated to a dose of 5×10¹⁴ ions/cm² using an argon GCIB accelerated using 30 kV acceleration voltage or were left un-irradiated, as controls. The mask employed was a non-contact shadow mask in proximity to the PTFE surface. The unmasked surface portions received the full GCIB dose, while the masked surface portions received no GCIB irradiation, thus serving as a control surface. Primary porcine fibroblast cells were harvested from fresh anterior ligament. The entire (irradiated and control portions) PTFE surfaces were seeded at an initial density of 5000 cells per cm² with the primary porcine fibroblast cells and allowed to attach for 24 hours in Dulbecco's Modified Eagle Medium nutrient mixture (DMEM) supplemented with 10% fetal bovine serum (FBS) and 1% penicillin/streptomycin and incubated in a humidified incubator at 37° C. Following 24 hours, media was removed and cells were briefly rinsed with 1× phosphate buffered saline and fixed in methanol pre-chilled at −20 degrees C. for 1 hour. Surfaces of the PTFE at the GCIB-irradiated portion, and at the non-GCIB-irradiated control portion were each imaged using a Hitachi TM-1000 scanning electron microscope. Results showed that there is a clear distinction between the cell attachment on the GCIB-irradiated portion versus the non-GCIB-irradiated portion of the PTFE surface.

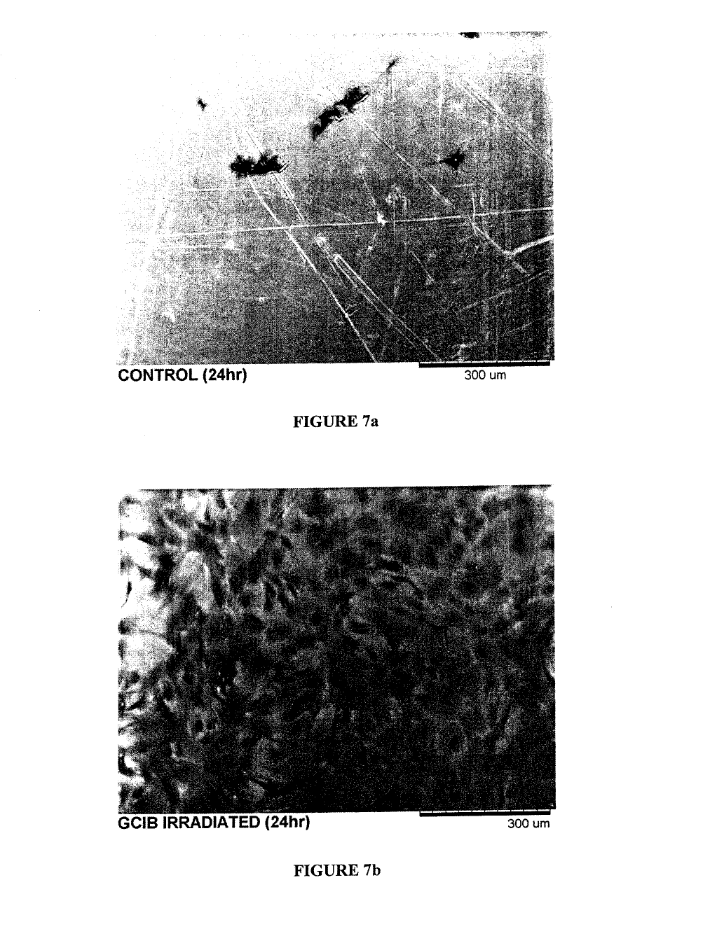

FIG. 7a is a scanning electron micrograph of the non-GCIB-irradiated control surface of the PTFE substrate taken 24 hours after seeding with cells. FIG. 7b is a scanning electron micrograph of the GCIB-irradiated surface of the PTFE substrate also taken 24 hours after seeding with cells (both following fixation).

FIG. 7a shows that cells attached to less than 1% of the non-GCIB-irradiated control portion of the PTFE surface.

FIG. 7b shows that cells attached to nearly 100% of the GCIB-irradiated portion of the PTFE surface.

This ability to impact cell attachment on a surface can be extremely useful in many applications where cell growth is desired in only restricted areas. Examples include PTFE cardiovascular stents that can be GCIB-irradiated on the luminal surface allowing re-endothelialization and maintaining intact (un-irradiated) PTFE surface on the abluminal surface to suppress smooth muscle growth and plaque formation; GCIB-irradiation of silicone rubber tubes to allow nerve regeneration; and other such.

### Amorphous Quartz Exemplary Embodiment

An amorphous quartz surface process is disclosed in a fifth exemplary embodiment. Amorphous quartz material is a material often employed in biological laboratory wares, also employed in fabricating medical objects intended for implantation into a mammal. Amorphous quartz is known to be a very favorably material for surface attachment and proliferation of cells. A clean and sterile amorphous quartz substrate was partially masked and then GCIB irradiated to a dose of 5×10¹⁴ ions/cm² using an argon GCIB accelerated using 30 kV acceleration voltage. The mask employed was a non-contact shadow mask in proximity to the quartz surface. The unmasked portion received the full GCIB dose, while the masked portion received no GCIB irradiation, thus serving as a control surface. Primary porcine fibroblast cells were harvested from fresh anterior ligament. The amorphous quartz surface was seeded at an initial density of 5,000 cells per cm² with the primary porcine fibroblast cells in Dulbecco's Modified Eagle Medium nutrient mixture (DMEM) supplemented with 10% fetal bovine serum (FBS) and 1% penicillin/streptomycin and incubated in a humidified incubator at 37° C. and 5% CO₂ in air. After 4 hours, the medium and non-adherent cells were then removed and replaced with fresh medium and incubation continued. The surface was viewed and imaged hourly for the first 4 hours and additionally at 6, 24, and 48 hours after initial seeding.

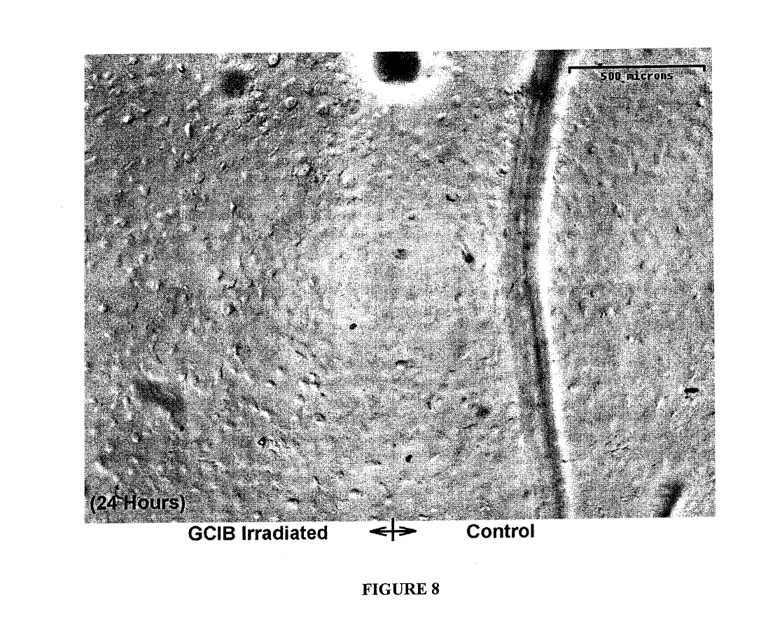

FIG. 8 is an optical micrograph of the partially masked amorphous quartz substrate taken at 24 hours after seeding with cells and viewed at the interface between the masked un-irradiated and the unmasked GCIB irradiated regions. The results show that fibroblast cells attach preferentially to the amorphous quartz surface regardless of whether or not the surface was GCIB irradiated or un-irradiated. The GCIB irradiated region is on the left side of FIG. 8 and the un-irradiated control region is on the right side of FIG. 8.

### Crystalline Sapphire Exemplary Embodiment

A (single crystal) crystalline sapphire surface improvement is disclosed in a sixth exemplary embodiment. A clean and sterile crystalline sapphire substrate was partially masked and then GCIB irradiated to a dose of 5×10¹⁴ ions/cm² using an argon GCIB accelerated using 30 kV acceleration voltage. The mask employed was a non-contact shadow mask in proximity to the sapphire surface. The unmasked portion received the full GCIB dose, while the masked portion received no GCIB irradiation, thus serving as a control surface. Primary porcine fibroblast cells were harvested from fresh anterior ligament. The crystalline sapphire surface was seeded at an initial density of 5,000 cells per cm² with the primary porcine fibroblast cells in Dulbecco's Modified Eagle Medium nutrient mixture (DMEM) supplemented with 10% fetal bovine serum (FBS) and 1% penicillin/streptomycin and incubated in a humidified incubator at 37° C. and 5% CO₂ in air. After 4 hours, the medium and non-adherent cells were then removed and replaced with fresh medium and incubation continued. The surface was viewed and imaged hourly for the first 4 hours and additionally at 6, 24, and 48 hours after initial seeding.

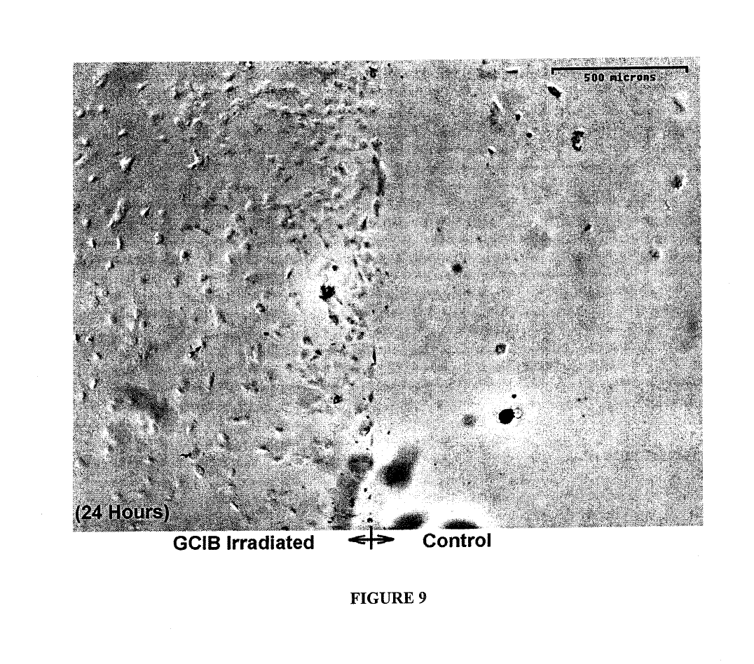

FIG. 9 is an optical micrograph of the partially masked crystalline sapphire substrate taken at 24 hours after seeding with cells and viewed at the interface between the masked un-irradiated and the unmasked GCIB irradiated regions. The GCIB irradiated region is on the left side of FIG. 9 and the un-irradiated control region is on the right side of FIG. 9. By comparing the un-irradiated and the GCIB irradiated regions, it is clear that the porcine fibroblast cells attach in greater numbers and proliferate better on the GCIB irradiated portion of the crystalline sapphire surface, compared to the un-irradiated (masked) portion.

It is believed that GCIB irradiation of a crystalline material like sapphire results in partial or complete amorphization of a very thin surface layer (a few tens of angstroms). Without wishing to be bound to any particular theory, it is possible that the amorphizing surface modification effected by the irradiation contributes to the improved cellular attachment and proliferation. Other possible mechanisms that may contribute to the improvement are increasing the surface wettability, hydrophilicity and/or modification of the surface charge state of the material.

### Polymer Filament/Polymer Fabric Exemplary Embodiment

Fabrics can be formed from polymer or co-polymer fibers by weaving, knitting, and/or by other non-woven techniques. Certain polymer fabrics (most notably polyethylene terephthalate) are particularly suitable fabrics for making vascular grafts. Fabric of woven polyethylene terephthalate (sometimes written as poly(ethylene terephthalate) and abbreviated PET, or PETE) fibers may also be referred to by one of its tradenames, Dacron, and is commonly employed as a material for fabricating vascular grafts. In a seventh exemplary embodiment, surface improvements are disclosed for a woven polyethylene terephthalate (PETE) fabric. Vascular grafts fabricated from PETE fabric are sometimes coated with a protein (such as collagen or albumin) to reduce blood loss and/or coated with antibiotics to prevent graft infection. Most strategies designed to reduce restenosis by the use of pharmacological or biological reagents involve direct inhibition of vascular smooth muscle cell proliferation on the fabric surface. However, as an alternative, smooth muscle cell proliferation may be indirectly inhibited by specific facilitation of re-endothelialization at injury and graft sites. In the past, re-endotheliaziation has often been slow or incomplete. In this embodiment we have evaluated GCIB irradiation of uncoated, woven PETE fabric material to show that it makes the material more bioactive and more suitable to facilitate re-endothelialization.

Woven PETE fabric was cut into 15 mm×30 mm pieces. The pieces were masked on one half and GCIB irradiated to a dose of 5×10¹⁴ ions/cm² using an argon GCIB accelerated using 30 kV acceleration voltage. The mask employed was a non-contact shadow mask in proximity to the PETE fabric surfaces and covering half of one side of each of the fabric pieces. The unmasked surface portions received the full GCIB dose, while the masked surface portions received no GCIB irradiation, thus serving as a control surface. The fabric pieces were placed in individual Petri dishes and live mouse endothelial cells (EOMA cell line) were seeded onto the entire (irradiated and control portions) PETE fabric surface at an initial density of 50,000 cells per fabric piece and allowed to attach for 24 hours in Dulbecco's Modified Eagle Medium nutrient mixture (DMEM) supplemented with 10% fetal bovine serum (FBS) and 1% penicillin/streptomycin during incubation in a humidified incubator at 37° C. Following 24 hours, media and un-adhered cells were removed. Methanol, pre-chilled at −20 degrees C. for 1 hour, was placed on the PETE fabric for 10 minutes to fix adherent cells. The fabric and adhered mouse endothelial cells were then imaged by scanning electron microscope. Surface regions of both the GCIB irradiated and unirradiated control portions of the PETE fabric with attached mouse endothelial cells were imaged using a Hitachi TM-1000 scanning electron microscope. Results showed that there is a clear distinction between the cell attachment on the GCIB-irradiated portion versus the non-GCIB-irradiated portion of the PETE woven fabric surface.

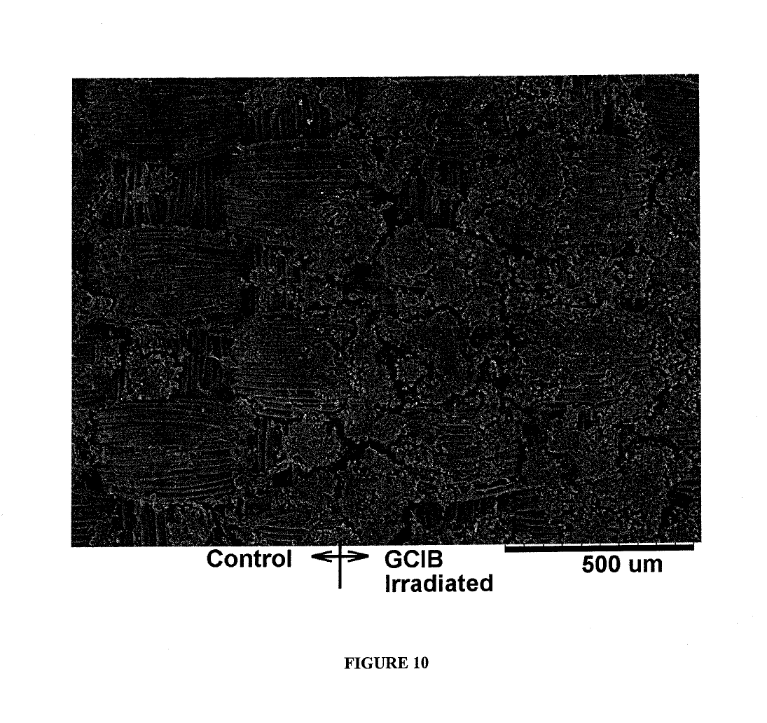

FIG. 10 is a scanning electron micrograph of a treated piece of PETE fabric surface made 24 hours after seeding with mouse endothelial cells (following methanol fixation). The portion of the PETE fabric on the left side of the image is the masked portion of the PETE fabric that was not irradiated prior to seeding. The portion of the PETE fabric on the right side of the image is the portion that received GCIB irradiation prior to seeding with cells.

FIG. 10 shows that re-endothelialization by mouse endothelial cells progressed significantly further on the GCIB irradiated portion of the PETE fabric than on the unirradiated control portion. EOMA cells preferentially adhered to the portion of PETE fabric that received GCIB irradiation.

In the several embodiments disclosed above, the method of this invention may further include combination with other previously known methods for improving the surfaces and/or for enhancing bioactivity and integration including, without limitation, sandblasting, acid etching, plasma spraying of coatings, CO₂ laser smoothing and various forms of cleaning, including mechanical, ultrasonic, plasma, and chemical cleaning techniques, the use of surfactants or the application of films or coatings having different wettability characteristics, UV treatment, UV and ozone treatment, covalently attaching poly(ethylene glycol) (PEG), and the application of protein products such as the antibody anti-CD34 and/or arginine-glycine-aspartate peptides (RGD peptides) and/or collagen and/or albumin. Such combinations are intended to be encompassed within the scope of the invention.

Although the invention has been described for exemplary purposes as employing titanium foil, glass, polystyrene, PTFE, quartz, sapphire, and PETE fabric surfaces, it is understood that objects for medical implant formed from titanium and/or titanium alloys (with or without oxide coatings), cobalt-chrome alloys, cobalt-chrome-molybdenum alloys, tantalum, tantalum alloys, various other metals and metal alloys, plastic or polymer or co-polymer materials including polyethylene and other inert plastics, solid resin materials, glassy materials, woven, knitted, and non-woven polymeric/co-polymeric fabrics, biological materials such as bone, collagen, silk and other natural fibers, various ceramics including titania, and other materials that may be suitable for the application and that are appropriately biocompatible. Although the invention has been described with respect to various embodiments and applications in the field of objects for medical implantation, it is understood by the inventors that its application is not limited to that field and that the concepts of GCIB irradiation of surfaces to make them more conducive to cellular growth, attachment, and attachment has broader application in fields that will be apparent to those skilled in the art. Such broader applications are intended to be encompassed within the scope of this invention. It should be realized that this invention is also capable of a wide variety of further and other embodiments within the spirit and scope of the invention and the claims.

What is claimed is:

1. A method of improving bioactivity of a surface of an implantable object, the method comprising:
forming a gas-cluster ion-beam in a reduced-pressure chamber;
introducing an object into the reduced-pressure chamber;
wherein said object is a medical prosthesis, a surgical implant, a surgical graft, a component of a medical prosthesis, a component of a surgical implant, a component of a surgical graft, or another object intended for implantation; and
irradiating at least a first portion of the surface of said object with a gas-cluster ion-beam.

2. The method of claim 1, further comprising cleaning said at least a portion of said surface prior to irradiating said at least a portion of said surface.

3. The method of claim 1, wherein the at least a first portion of the surface comprises a metal, an oxide, a metal alloy, a plastic, a polymer, a copolymer, a solid resin, a rubber, glass, quartz, a ceramic, sapphire, a glassy material, titanium, titania, an alloy of titanium, a cobalt-chrome alloy, a cobalt-chrome-molybdenum alloy, tantalum, a tantalum alloy, a biological material, a polymeric or copolymeric fabric, a silicone, bone, collagen, silk, or a natural fiber material.

4. The method of claim 1, wherein said object comprises a fabric.

5. The method of claim 1, wherein the surface of the object comprises an amorphous material.

6. The method of claim 1, wherein the surface of the object comprises a crystalline material.

7. The method of improving bioactivity of a surface of biological laboratory ware, the method comprising:
forming a gas-cluster ion-beam in a reduced-pressure chamber;
introducing an object into the reduced-pressure chamber;
wherein said object is an item of biological laboratory ware; and
irradiating at least a first portion of the surface of said object with a gas-cluster ion-beam.

8. The method of claim 7, wherein at least a second portion of the surface of said object is not irradiated by a gas-cluster ion-beam.

9. A method of attaching cells to an object, the method comprising:
selecting at least a portion of a surface of an object;
forming a gas-cluster ion-beam in a reduced-pressure chamber;
introducing said object into said reduced-pressure chamber;
irradiating said at least a portion of said surface with said gas-cluster ion-beam;
removing said object from said reduced-pressure chamber; and
exposing said at least a portion of said surface to living cells.

10. The method of claim 9, wherein said exposing is performed for a period of time necessary for initiating cell growth on said at least a portion of said surface.

11. The method of claim 9, further comprising cleaning said at least a portion of said surface prior to irradiating said at least a portion of said surface.

12. The method of claim 9, wherein said at least a portion of said surface comprises a material selected from the group consisting of a metal, an oxide, a metal alloy, a plastic, a polymer, a copolymer, a solid resin, a rubber, glass, quartz, a ceramic, sapphire, a glassy material, titanium, titania, a titanium alloy, alumina, zirconium, an zirconium alloy, zirconia, a cobalt-chrome alloy, a cobalt-chrome-molybdenum alloy, tantalum, a tantalum alloy, a biological material, a polymeric fabric, a copolymeric fabric, a silicone, bone, collagen, silk, and a natural fiber.

13. The method of claim 9, wherein said object is a medical prosthesis, a surgical implant, a surgical graft, a component of a medical prosthesis, a component of a surgical implant, or a component of a surgical graft, or another object intended for implant into a body of a mammal.

14. The method of claim 13, wherein the surgical graft comprises a woven, knitted, or non-woven fabric.

15. The method of claim 9, wherein the surface of the object comprises an amorphous material.

16. The method of claim 9, wherein the surface of the object comprises a crystalline material.

17. The method of claim 9, wherein the object is an item of biological laboratory ware.

18. The method of claim 9, wherein the object is an environmental testing device.

19. The method of claim 1, wherein at least a second portion of a surface of said object is not irradiated by a gas-cluster ion-beam.

20. A method of preparing an object for medical implantation, said method comprising:
selecting at least a portion of a surface of an object;
wherein said object is a medical implant;
forming a gas-cluster ion-beam in a reduced-pressure chamber;
introducing the object into the reduced-pressure chamber; and
irradiating the selected at least a portion with the gas-cluster ion-beam to increase the bioactivity of the at least a portion.

21. The method of claim 20, further comprising the step of:
attaching and growing cells on at least the irradiated portion of the object in vitro, prior to medical implantation.

22. A article with attached cells made by a method comprising the steps of
selecting at least a portion of a surface of an object for attaching cells;
forming a gas-cluster ion-beam in a reduced-pressure chamber;
introducing said article into said reduced-pressure chamber;
irradiating said at least a portion of said surface with the gas-cluster ion-beam;
removing said object from said reduced-pressure chamber; and
exposing said at least a portion of said surface to living cells.

23. An article for medical implantation made by a method comprising:
selecting at least a portion of a surface of a medical implant;
forming a gas-cluster ion-beam in a reduced-pressure chamber;
introducing said implant into said reduced-pressure chamber; and
irradiating said at least a portion of said surface with the gas-cluster ion-beam to increase the bioactivity of said at least a portion of said surface.
